(19) United States
(12) Patent Application Publication
(10) Pub. No.: US 2021/0295983 A1
(43) Pub. Date: Sep. 23, 2021

(54) MEDICAL INFORMATION MANAGEMENT APPARATUS, MEDICAL INFORMATION MANAGEMENT SYSTEM, STORAGE MEDIUM, AND MEDICAL INFORMATION MANAGEMENT METHOD

(71) Applicant: Konica Minolta, Inc., Tokyo (JP)

(72) Inventor: Seiji NOMURA, Tokyo (JP)

(21) Appl. No.: 17/201,547

(22) Filed: Mar. 15, 2021

(30) Foreign Application Priority Data
Mar. 17, 2020 (JP) 2020-045908

(51) Int. Cl. G16H 40/20

(52) U.S. Cl. CPC G16H 40/20

(57) ABSTRACT

A medical information management apparatus includes a storage and a hardware processor. The storage stores medical information, doctor identification information, and a confirmation status of the medical information that are associated with each other. The hardware processor obtains doctor identification information of a doctor that operates a display. In displaying target medical information on the display, in response to the target medical information being associated with the obtained doctor identification information in the storage, the hardware processor gives confirmation authority to the doctor, the confirmation authority being authority to determine a confirmation status of the target medical information in the storage as having been confirmed. The hardware processor provides the display with a confirmation instruction receiver for determining the confirmation status of the target medical information as having been confirmed.

### CROSS-REFERENCE TO RELATED APPLICATIONS

The present invention claims priority under 35 U.S.C. § 119 to Japanese Patent Application No. 2020-045908 filed on Mar. 17, 2020, the entire content of which is incorporated herein by reference.

### BACKGROUND

### Technological Field

The present disclosure relates to a medical information management apparatus, a medical information management system, a storage medium, and a medical information management method.

### Description of Related Art

Medical information, such as medical images and reports, has been electronized and managed as electronic data in the medical field. When a doctor requests an image interpretation doctor to interpret a medical image, the doctor who requested the interpretation confirms an image interpretation report prepared by the image interpretation doctor and utilizes the report for diagnosis. Thus, medical information may be supposed to be confirmed by a specific doctor.

There is known a technology of identifying an individual and preventing falsification of electronic data in digitally signing the electronic data. For example, JP2005-094703A discloses a technology to determine whether or not to allow a person to sign digitally, based on the authority given to an owner of a secret key. According to the technology, authority information is obtained from identification information for identifying an owner of the secret key. The authority information indicates presence/absence of qualification and the level of the qualification. Based on the obtained authority information, determination is made on whether or not the owner of the secret key has the authority to sign digitally.

### SUMMARY

According to JP2005-094703A, however, the presence/absence of the authority to sign digitally is determined based on the qualification in the authority information. When the technology is used in confirming medical information, a doctor not supposed to confirm the medical information may confirm the medical information.

The known method thus may not secure appropriate management of medical information.

The present invention has been conceived in view of the above issues. Objects of the present invention include: preventing a doctor not supposed to confirm medical information from confirming the medical information; and managing the medical information securely.

To achieve at least one of the abovementioned objects, according to an aspect of the present invention, there is provided a medical information management apparatus, including: a storage that stores medical information, doctor identification information, and a confirmation status of the medical information that are associated with each other; and a hardware processor that: obtains doctor identification information of a doctor that operates a display; in displaying target medical information on the display, in response to the target medical information being associated with the obtained doctor identification information in the storage, gives confirmation authority to the doctor, the confirmation authority being authority to determine a confirmation status of the target medical information in the storage as having been confirmed; and provides the display with a confirmation instruction receiver for determining the confirmation status of the target medical information as having been confirmed.

To achieve at least one of the abovementioned objects, according to another aspect of the present invention, there is provided a medical information management system, including: a storage that stores medical information, doctor identification information, and a confirmation status of the medical information that are associated with each other; a display; and a hardware processor that: obtains doctor identification information of a doctor that operates the display; in displaying target medical information on the display, in response to the target medical information being associated with the obtained doctor identification information in the storage, gives confirmation authority to the doctor, the confirmation authority being authority to determine a confirmation status of the target medical information in the storage as having been confirmed; and provides the display with a confirmation instruction receiver for determining the confirmation status of the target medical information as having been confirmed.

To achieve at least one of the abovementioned objects, according to another aspect of the present invention, there is provided a non-transitory computer readable storage medium storing a program to cause a computer of a medical information management apparatus that includes a storage storing medical information, doctor identification information, and a confirmation status of the medical information that are associated with each other to function as a hardware processor that: obtains doctor identification information of a doctor that operates the display; in displaying target medical information on the display, in response to the target medical information being associated with the obtained doctor identification information in the storage, gives confirmation authority to the doctor, the confirmation authority being authority to determine a confirmation status of the target medical information in the storage as having been confirmed; and provides the display with a confirmation instruction receiver for determining the confirmation status of the target medical information as having been confirmed.

To achieve at least one of the abovementioned objects, according to another aspect of the present invention, there is provided a medical information management method for a medical information management apparatus that includes a storage storing medical information, doctor identification information, and a confirmation status of the medical information that are associated with each other, the method including: obtaining doctor identification information of a doctor that operates a display; in displaying target medical information on the display, in response to the target medical information being associated with the obtained doctor identification information in the storage, giving confirmation authority to the doctor, the confirmation authority being authority to determine a confirmation status of the target medical information in the storage as having been confirmed; and providing the display with a confirmation instruction receiver for determining the confirmation status of the target medical information as having been confirmed.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 shows a structure of a medical information management system according to the present invention;

FIG. 2 is a block diagram of functional components of a medical information management apparatus;

FIG. 3 shows a data structure of a facility information table, a department information table, a user information table, and a user attribution table;

FIG. 4 shows a data structure of a patient information table, a report information table, a report confirmation history table, a report reference history table, and a medical image management table;

FIG. 5 is a block diagram of functional components of a client terminal;

FIG. 6 is a flowchart of a report confirmation status managing process to be performed by the medical information management apparatus;

FIG. 7 is an example of a search window;

FIG. 8 is an example of an unconfirmed report list window of a log-in user;

FIG. 9 is an example of an examination/report list window;

FIG. 10 is an example of an unconfirmed report list window of a selected patient;

FIG. 11 is a flowchart of a report window displaying process;

FIG. 12 is a flowchart of an authority determining process; and

FIG. 13 is an example of a report window.

### DETAILED DESCRIPTION OF THE EMBODIMENTS

Hereinafter, one or more embodiments of a medical information management system according to the present invention will be described with reference to the drawings.

Although various technically preferred limitations for carrying out the present invention are imposed on the embodiment below, the technical scope of the present invention is not limited to the embodiment or drawings.

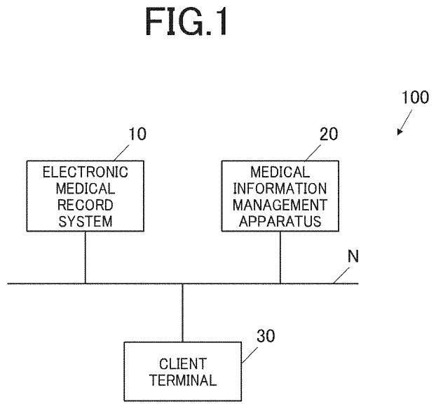

FIG. 1 shows an example of a structure of a medical information management system 100.

As shown in FIG. 1, the medical information management system 100 includes an electronic medical record system 10, a medical information management apparatus 20, and a client terminal(s) 30. These apparatuses are connected to each other for data exchange over a communication network N, such as a local area network (LAN) or a wide area network (WAN). The apparatuses constituting the medical information management system 100 conform to Health Level Seven (HL7) standard and/or Digital Image and Communications in Medicine (DICOM) standard, and communicate with each other according to these standards. The medical information management system 100 includes multiple client terminals 30.

The electronic medical record system 10 generates electronic medical record information and order information. The electronic medical record information includes information on medical treatments and results of diagnosis. The order information is for requesting creation of reports on examinations of patients and medical images.

The medical information management apparatus 20 is a computer that manages medical information. Examples of the medical information management apparatus 20 include a computer of a picture archiving and communication system (PACS).

Medical information is information on medical services. The medical information preferably includes findings on a patient, diagnosis details, developments, and/or examination results. The medical information may further include medical reports and various kinds of information that constitute the medical reports, such as patient information, examination information, medical images (examination images, images on measurement results), measurement results, and findings.

A medical report is a report on findings on a patient, diagnosis details, developments, and/or examination results. The medical report may further include, for example, patient information, examination information, medical images (examination image, image on measurement results), enlarged or reduced images of the medical images, measurement results, findings, and header/footer. In the medical report, the region in which each item is entered/pasted and arrangements of the regions may be specified as a format.

Examples of the medical report include an image interpretation report on medical image data and an examination report.

An image interpretation report is a report prepared by an image interpretation doctor in the radiology department at the request of a doctor, for example. The image interpretation report includes findings on medical images taken with an imaging device, such as presence/absence of abnormalities, conditions of the patient, and comments on a disease. Examples of the imaging device include various modalities, such as a computed tomography (CT) and a magnetic resonance imaging (MRI)). The image interpretation report may further include the type, anatomical classification, extent, and conditions of a lesion, and the conclusion on the lesion.

An examination report is a report on an examination of the patient. Examples of the examination report include a sample report on a sample (e.g., blood) taken from the patient, bacterial report on bacteria in the patient, and a physical report on physical information of the patient.

The patient information includes various kinds of information on the patient, for example, the patient's name, patient ID, date of birth, and sex.

The examination information includes information on an examination, such as examination items, results (numerical values), combination of the items and results, examination date and time, notes on the examination, and the instance unique identifier (UID) of the examination.

A medical image shows results of imaging and/measuring a human body as an image for medical treatment and medical science. The medical image is used for diagnosis. Examples of the medical image include an x-ray image, MRI image, CT image, and pathologic image. The medical image may be a schema (manually drawn picture) that includes as much information as the abovementioned images.

The measurement result shows results of various medical measurements on the basis of the examination information, medical images, and so forth. The measurement result includes results of measuring cardiothoracic ratio and visceral fat areas, for example.

Findings include various decisions and opinions on the results of asking questions of the patient and examining the body and medical images. The findings include findings on the body, for example. The findings in the medical report include decisions and opinions on the results of examining the medical image.

The header and footer may include the logo, name, address, phone number, and e-mail address of a hospital, for example.

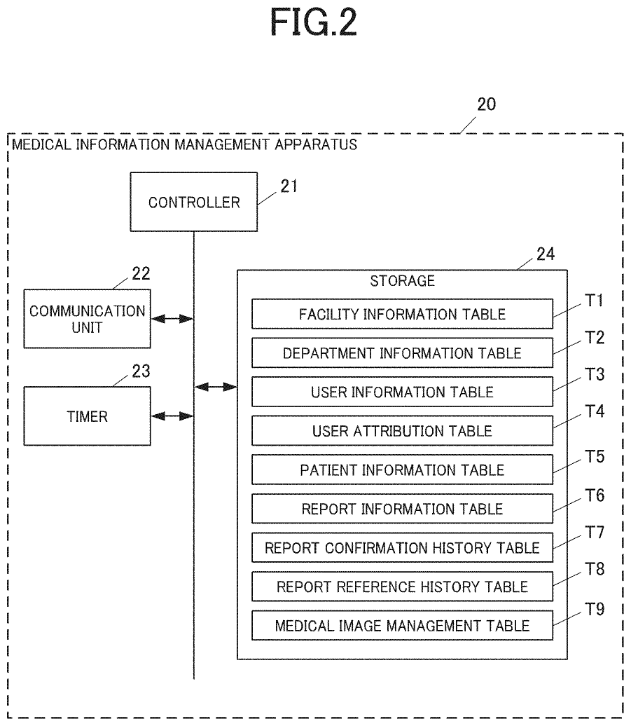

FIG. 2 shows functional components of the medical information management apparatus 20.

As shown in FIG. 2, the medical information management apparatus 20 includes a controller 21 (hardware processor), a communication unit 22, a timer 23, and a storage 24. These components are connected via a bus.

The controller 21 includes a central processing unit (CPU), a read only memory (ROM), and a random access memory (RAM). The controller 21 centrally controls processing operation of the components of the medical information management apparatus 20. The CPU reads various processing programs stored in the ROM, loads them into the RAM, and performs various processes in cooperation with the programs.

The communication unit 22 includes a network interface. The communication unit 32 transmits and receives data to and from external apparatuses connected over the communication network N.

The timer 23 includes a timer circuit (real time clock (RTC)). The timer 23 keeps current date and time with the timer circuit and outputs the date and time to the controller 21.

The storage 24 includes a hard disk drive (HDD) and/or a nonvolatile semiconductor memory and stores various kinds of data. For example, the storage 24 stores medical information including medical reports and medical images generated in examinations on patients.

The storage 24 further stores a facility information table T1, department information table T2, user information table T3, user attribution table T4, patient information table T5, report information table T6, report confirmation history table T7, report reference history table T8, and medical image management table T9.

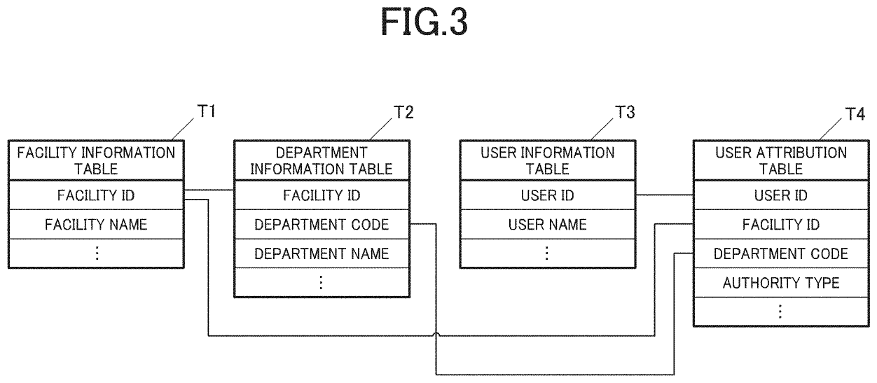

FIG. 3 shows the data structure of the facility information table T1, department information table T2, user information table T3, and user attribution table T4.

The facility information table T1 stores information on medical facilities. In the facility information table T1, each medical facility is associated with a facility ID, facility name, and so forth. The facility ID is identification information given to the medical facility. The facility name is the name of the facility.

The department information table T2 stores information on medical departments in a medical facility. In the department information table T2, each department of the medical facility is associated with the facility ID of the facility to which the department belongs, department code, department name, and so forth. The department code is identification information given to the department. The department name is the name of the department.

The user information table T3 stores information on users (doctors) who use the medical information management apparatus 20. In the user information table T3, each user is associated with a user ID, user name, and so forth. The user ID is identification information given to the user. The user name is displayed when the user uses the medical information management apparatus 20.

The user attribution table T4 stores attribution information of users (e.g., departments and positions). In the user attribution table T4, each user is associated with a user ID (user ID of the target user), facility ID of a medical facility to which the user belongs, department code of a department to which the user belongs, authority type, and so forth. The authority types correspond to the authorities set for users. In this embodiment, the authority types are 0: none, 1: resident doctor, 2: doctor, 3: department head, and 4: administrator. A resident doctor is a doctor who is in a clinical training period. A department head is a doctor in charge of the department to which the doctor belongs. An administrator is an administrator of the medical information management apparatus 20.

There may be a case where a doctor belongs to multiple departments or a case where different departments have different authority types. To deal with these cases, the user attribution table T4 can store multiple pieces of attribution information for each user.

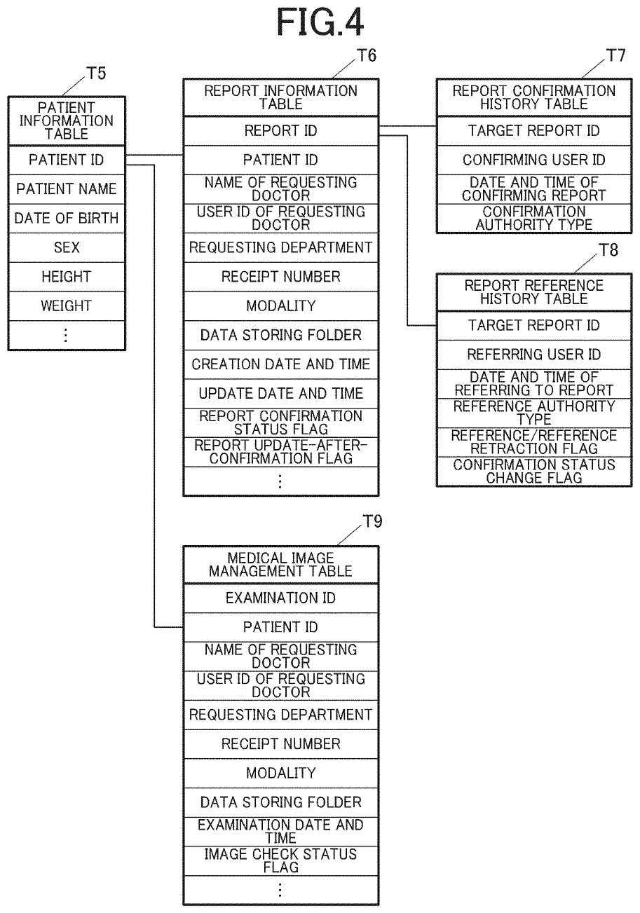

FIG. 4 is a data structure of the patient information table T5, report information table T6, report confirmation history table T7, report reference history table T8, and medical image management table T9.

The patient information table T5 stores information on patients. In the patient information table T5, each patient is associated with a patient ID, patient name, date of birth, sex, height, weight, and so forth. The patient ID is identification information given to the patient. The patient name, date of birth, sex, height, and weight are the name, date of birth, sex, height, and weight of the patient.

The report information table T6 stores information on medical reports. In the report information table T6, each report is associated with a report ID, patient ID, name of requesting doctor, user ID of requesting doctor, requesting department, receipt number, modality, data storing folder, creation date and time, update date and time, report confirmation status flag, and report update-after-confirmation flag.

The report ID is identification information given to the medical report.

The patient ID is the patient ID of a patient relating to the medical report.

The name of requesting doctor is the name of a doctor (requesting doctor) who requested creation of the medical report.

The user ID of requesting doctor is the user ID of the doctor who requested creation of the medical report.

The requesting department is a medical department that requested creation of the medical report.

The requesting department is normally a department to which the requesting doctor belongs.

The receipt number is given to the creation of the medical report.

The modality is an imaging device that is used in the examination targeted by the medical report.

The data storing folder indicates a folder (storage place) in which data of the medical report (e.g., PDF file) is stored.

The creation date and time is the date and time at which the medical report was created.

The update date and time is the date and time at which the medical report was updated.

The report confirmation status flag indicates the confirmation status of the medical report.

The report confirmation status flag indicates 0: unconfirmed (unread), 1: confirmed (read), or 2: being confirmed. The default value of the report confirmation status flag is “0”. When the medical report is updated after having been confirmed, the report confirmation status flag returns to “0: unconfirmed”.

The report update-after-confirmation flag indicates whether or not the medical report is updated after having been confirmed. The default value of the report update-after-confirmation flag is “0”. When the medical report is updated after having been confirmed, the report update-after-confirmation flag becomes “1”.

In the report information table T6, medical information (report ID) is associated with identification information on a doctor (user ID of requesting doctor) and the confirmation status of the medical information (report confirmation status flag).

The report confirmation history table T7 is a table for recording whether or not medical reports registered in the report information table T6 have been confirmed. In the report confirmation history table T7, the target report ID is associated with a confirming user ID, report confirmation date and time, and confirmation authority type.

The target report ID is the report ID of the medical report as a target.

The confirming user ID is the user ID of a user who confirmed the medical report (who pressed a confirmation button).

The report confirmation date and time is the date and time at which the medical report was determined as having been confirmed. The confirmation authority type is the authority type of the confirming user who confirmed the medical report.

The report reference history table T8 is for managing the history of referring to the medical reports registered in the report information table T6. In the report reference history table T8, the target report ID is associated with a referring user ID, report reference date and time, reference authority type, reference/reference retraction flag, and confirmation status change flag.

The target report ID is the report ID of the target medical report.

The referring user ID is the user ID of the user (referring user) who referred to the medical report (who displayed the medical report).

The report reference date and time is the date and time at which the medical report was referred to.

The reference authority type is the authority type of the referring user who referred to the medical report.

The reference/reference retraction flag indicates operation by users. The reference/reference retraction flag has values of 0: no operation, 1: referred, and 2: reference retracted.

The confirmation status change flag indicates changes of the report confirmation status flag in the report information table T6. The confirmation status change flag has values of 0: unchanged, 1: changed to “confirmed”, and −1: confirmation retracted.

The medical image management table T9 is for managing medical images stored in the storage 24. In the medical image management table T9, each examination is associated with an examination ID, patient ID, name of requesting doctor, user ID of requesting doctor, requesting department, receipt number, modality, data storing folder, examination date and time, and image check status flag.

The examination ID is the examination ID of the target examination.

The patient ID is the patient ID of the patient as a target of the examination.

The name and user ID of requesting doctor are the names and user ID of the doctor who requested the examination (imaging).

The requesting department is the medical department that requested the examination.

The receipt number is given to the examination.

The modality is the imaging device that is used in the target examination.

The data storing folder indicates a folder (storage place) in which data of the medical image relating to the examination is stored.

The examination date and time is the date and time at which the examination (imaging) was performed.

The image check status flag indicates the check status of the medical image that relates to the examination. The image check status flag indicates 0: unchecked, 1: checked, or 2: being checked. The default value of the image check status flag is “0”.

The controller 21 obtains staff information (user information and user attribution) of each medical facility from the electronic medical record system 10, and registers the obtained staff information in the user information table T3 and the user attribution table T4.

When the electronic medical record system 10 issues order information to order a medical report, the controller 21 obtains the order information (patient ID, name of requesting doctor, user ID of requesting doctor, requesting department, receipt number, and modality) from the electronic medical record system 10 via the communication unit 22. The controller 21 then registers the obtained order information in the report information table T6. More specifically, the controller 21 adds a new record in the report information table T6. For the new record, the controller 21 generates and registers a new report ID associated with the patient ID, name of requesting doctor, user ID of requesting doctor, requesting department, receipt number, and modality.

When a medical report is created in a terminal device used by an image interpretation doctor, the controller 21 obtains the medical report including the report ID and receipt number via the communication unit 22. The controller 21 stores the obtained medical report in the storage 24 and registers the information on the medical report in the report information table T6. More specifically, the controller 21 identifies a record in the report information table T6, based on the report ID and/or the receipt number of the medical report. The controller 21 then registers, for the identified record, the data storing folder and the creation date and time. Herein, the report confirmation status flag is “0: unconfirmed”, and the report update-after-confirmation flag is “0: default value”. In response to obtaining the medical report, the controller 21 notifies the electronic medical record system 10 that the medical report was created.

The controller 21 obtains identification information (user ID) of a doctor who operates the client terminal 30 (display). The controller 21 thus functions as an obtaining unit.

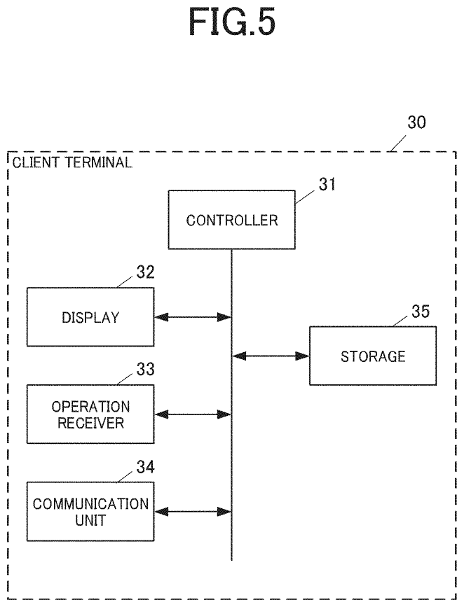

The controller 21 displays target medical information on the display 32 (in FIG. 5) of the client terminal 30. When the target medical information is associated with the identification information of the doctor operating the client terminal 30 in the report information table T6, the controller 21 gives confirmation authority to the doctor. The confirmation authority is the authority to determine the confirmation status (report confirmation status flag) of the target medical information as having been confirmed in the report information table T6 of the storage 24. More specifically, the controller 21 provides the client terminal 30 with a confirmation instruction receiver to determine the confirmation status of the target medical information as having been confirmed in the report information table T6.

The confirmation instruction receiver is to record that the medical information is confirmed. Examples of the confirmation instruction receiver include a button, checkbox, digital signature and/or entry section. A button may be displayed to be clickable on a display screen of a display (e.g., display 32 of the client terminal 30).

A checkbox is a small box displayed on the display screen of the display to switch between two statuses and select one of the statuses (e.g., on and off). For digital signature, the display screen of the display performs display such that a user can make an instruction to sign digitally. An entry section is displayed on the display screen of the display for entering specific words for confirmation. For example, the entry section may be a section for entering a personal identification ID.

In the present disclosure, “determining the confirmation status of the medical information as having been confirmed” with the confirmation instruction receiver includes (i) making instruction to determine the confirmation status of the medical information as having been confirmed and (ii) recording the confirmation status of the medical information as having been confirmed.

As the confirmation instruction receiver for determining the confirmation status of the target medical information as having been confirmed, the controller 21 displays, on the client terminal 30, a confirmation button. The confirmation button can be pressed by operating the operation receiver 33 (shown in FIG. 5).

The confirmation button is pressed after a doctor confirms the medical information. When the confirmation button is pressed, the controller 21 records that the medical information is confirmed. The confirmation button is displayed so as to be clickable on the display screen of the display, for example.

When the confirmation button displayed on the client terminal 30 is pressed by operating the operation receiver 33, the controller 21 changes the confirmation status (report confirmation status flag) of the target medical information as having been confirmed in the report information table T6 in the storage 24. The controller 21 then prohibits the confirmation button from being pressed again.

When the medical information is confirmed, the controller 21 notifies the electronic medical record system 10 that the medical information is confirmed.

When the target medical information is not associated with the identification information of the doctor operating the client terminal 30 in the report information table T6 of the storage 24, the controller 21 displays, on the client terminal 30, the confirmation button that cannot be pressed by operating the operation receiver 33.

When the target medical information is handled emergently, the controller 21 gives confirmation authority to the doctor even if the target medical information is not associated with the identification information of the doctor operating the client terminal 30 in the report information table T6 of the storage 24. Whether or not to emergently handle medical information may be determined based on departments that requested creation of the medical information. For example, the general department for emergency may be determined as a specific department for which medical information is handled emergently. Entities to which confirmation authority is given in emergency may be limited. For example, resident doctors may be excluded.

When the controller 21 gives confirmation authority to a doctor because the target medical information is emergently handled, the controller 21 displays a warning on the display 32 of the client terminal 30.

The controller 21 displays, on the display 32 of the client terminal 30, a list of unconfirmed medical information the confirmation authority of which is given to a doctor operating the client terminal 30. In displaying the list, the controller 21 excludes, from the list, medical information the confirmation authority of which is given to the doctor because the medical information is handled emergently.

The controller 21 also gives confirmation authority to a doctor operating the client terminal 30, when the operating doctor is the head of a department to which a doctor indicated by identification information associated with the target information belongs.

The controller 21 records history of determining the confirmation status of the target medical information as having been confirmed with the confirmation instruction receiver. More specifically, when the report confirmation status flag of a medical report is changed to “1: confirmed” in the report information table T6 of the storage 24, the controller 21 records the target report ID, confirming user ID, confirmation date and time, and confirmation authority type of the target report in the report confirmation history table T7.

The controller 21 permits a doctor who is given confirmation authority of medical information stored in the storage 24 to retract the confirmation of the medical information.

The client terminal 30 is a computer used by doctors, such as a personal computer. Doctors review medical information including medical reports on the client terminals 30.

FIG. 5 shows functional components of the client terminal 30.

As shown in FIG. 5, the client terminal 30 includes a controller 31, a display 32, an operation receiver 33, a communication unit 34, and a storage 35. These components are connected via a bus.

The controller 31 includes a CPU, ROM, and RAM and centrally controls processing operation of the components of the client terminal 30. The CPU reads out various programs stored in the ROM, loads the read programs into the RAM, and performs various processes in cooperation with the programs.

The display 32 includes a monitor, such as a liquid crystal display (LCD), and displays various windows/screens in accordance with instructions of display signals input by the controller 31.

The operation receiver 33 includes: a keyboard including cursor keys, character and number entry keys, and various function keys; and a pointing device, such as a mouse. The operation receiver 33 outputs operation signals input with the keyboard or the mouse.

The communication unit 34 includes a network interface. The communication unit 34 sends and receives data to and from external apparatuses connected over the communication network N.

The storage 35 includes an HDD and/or a nonvolatile semiconductor memory and stores various data.

Next, operation of the medical information management system 100 is described.

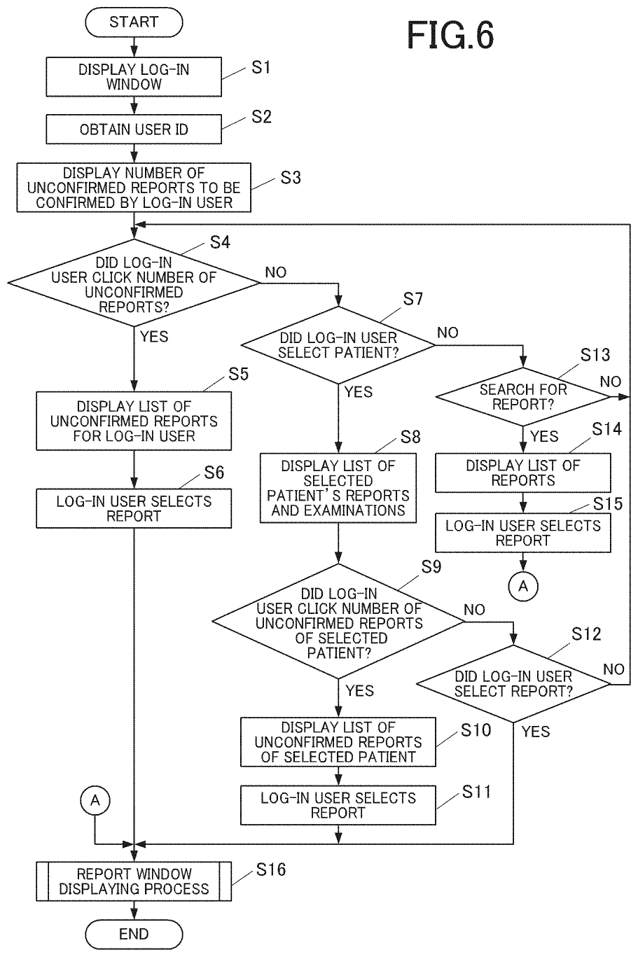

FIG. 6 is a flowchart of a report confirmation status managing process to be performed by the medical information management apparatus 20. The CPU of the controller 21 performs the process through software processing in cooperation with a control program(s) stored in the ROM.

When an operator of the client terminal 30 accesses the medical information management apparatus 20 by operating the operation receiver 33, the controller 21 of the medical information management apparatus 20 sends display data to the client terminal 30 via the communication unit 22 (Step S1). The display data is data for displaying a log-in window.

On the display 32 of the client terminal 30, the log-in window is displayed. The log-in window has a user ID input section.

When the operator of the client terminal 30 (log-in user) inputs his/her user ID with the operation receiver 33, the controller 21 of the medical information management apparatus 20 obtains the input user ID via the communication unit 22 (Step S2).

The controller 21 of the medical information management apparatus 20 causes the display 32 of the client terminal 30 to display the number of unconfirmed reports that need to be confirmed by the log-in user (Step S3). More specifically, the controller 21 extracts, from the report information table T6 in the storage 24, a record(s) in which (i) the field of “user ID of requesting doctor” has the user ID of the log-in user and (ii) “report confirmation status flag” is “0: unconfirmed”. The medical reports that correspond to the extracted records are the unconfirmed reports of the log-in user, and the number of extracted records is the number of unconfirmed reports of the log-in user.

When the log-in user is a department head of a department, the display 32 of the client terminal 30 displays the number of unconfirmed reports requested by the department. More specifically, the controller 21 of the medical information management apparatus 20 refers to the user attribution table T4 in the storage 24. When the user ID of the log-in user is associated with the authority type of “3: department head”, the controller 21 obtains the department code associated with the user ID of the log-in user. The controller 21 then extracts, from the report information table T6, a record(s) in which (i) the field of “requesting department” is the department to which the log-in user belongs (the department that corresponds to the department code obtained from the user attribution table T4) and (ii) report confirmation status flag is “0: unconfirmed”.

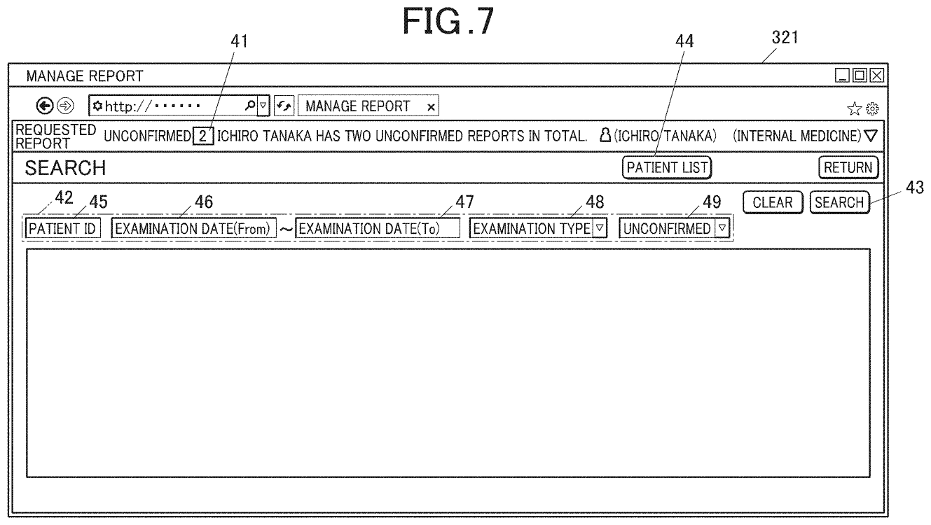

FIG. 7 is an example of a search window 321 displayed on the display 32 of the client terminal 30. The search window 321 includes a user-unconfirmed-report-number icon 41, a search-condition specifying region 42, a search button 43, and a patient list button 44.

The user-unconfirmed-report-number icon 41 indicates the number of unconfirmed reports of the log-in user. When the user presses the user-unconfirmed-report-number icon 41, a list of unconfirmed reports of the log-in user is displayed.

The search-condition specifying region 42 includes a patient-ID specifying section 45, an examination-date specifying sections 46, 47, an examination-type specifying section 48, and a confirmation-status specifying section 49. In the patient-ID specifying section 45, a patient ID is input as a search condition. In the examination-date specifying sections 46, 47, the range of examination dates (from when to when) are specified as a search condition. In the examination-type specifying section 48, the type of examination (e.g., modality) for which reports are made is specified as a search condition. In the confirmation-status specifying section 49, the confirmation status of medical reports (confirmed, unconfirmed, or all) is specified as a search condition.

The search button 43 is a button for making instructions to perform search according to the search conditions specified in the search-condition specifying region 42.

The patient list button 44 is a button for displaying the list of patients.

Next, the controller 21 of the medical information management apparatus 20 determines whether or not the number of unconfirmed reports for the log-in user (user-unconfirmed-report-number icon 41) displayed on the display 32 has been clicked with the operation receiver 33 of the client terminal 30 (Step S4).

When determining that the number of unconfirmed reports for the log-in user has been clicked (Step S4: YES), the controller 21 of the medical information management apparatus 20 causes the display 32 of the client terminal 30 to display the list of unconfirmed reports for the log-in user (Step S5).

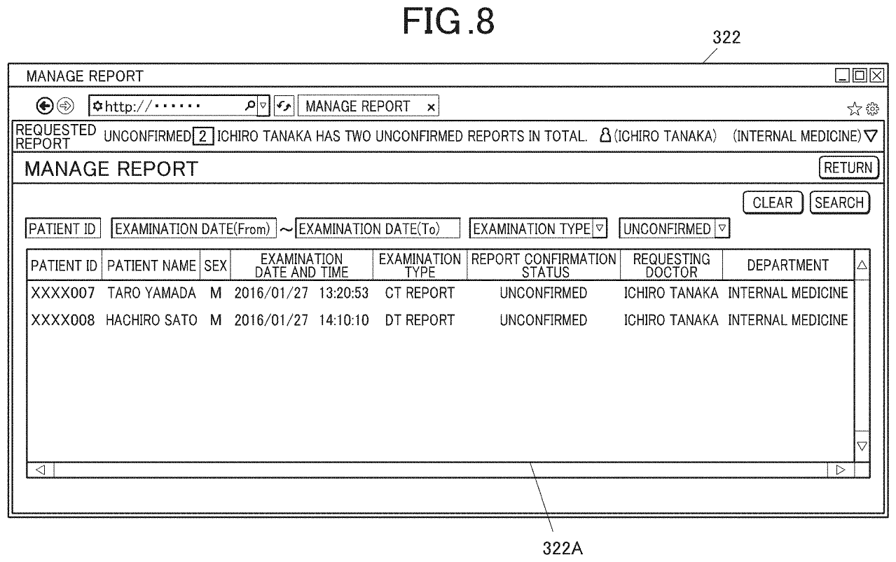

FIG. 8 is an example of the unconfirmed report list window 322 for the log-in user, which is displayed on the display 32 of the client terminal 30. The unconfirmed report list window 322 for the log-in user shows an unconfirmed report list 322A that includes unconfirmed reports requested by the log-in user.

When the operator selects any of the unconfirmed reports in the unconfirmed report list of the log-in user (Step S6), the controller 21 of the medical information management apparatus 20 obtains the report ID of the selected report via the communication unit 22.

When determining that the number of unconfirmed reports for the log-in user has not been clicked (Step S4: NO), the controller 21 of the medical information management apparatus 20 determines whether or not a patient has been selected in a patient list with the operation receiver 33 of the client terminal 30 (Step S7). For example, when the patient list button 44 is pressed in the search window 321 shown in FIG. 7, the display 32 of the client terminal 30 displays a patient list to allow the operator to select a patient in the patient list.

When determining that a patient has been selected with the operation receiver 33 of the client terminal 30 (Step S7: YES), the controller 21 of the medical information management apparatus 20 obtains the patient ID of the selected patient via the communication unit 22.

The controller 21 of the medical information management apparatus 20 then causes the display 32 of the client terminal 30 to display a list of examinations and reports of the selected patient (Step S8).

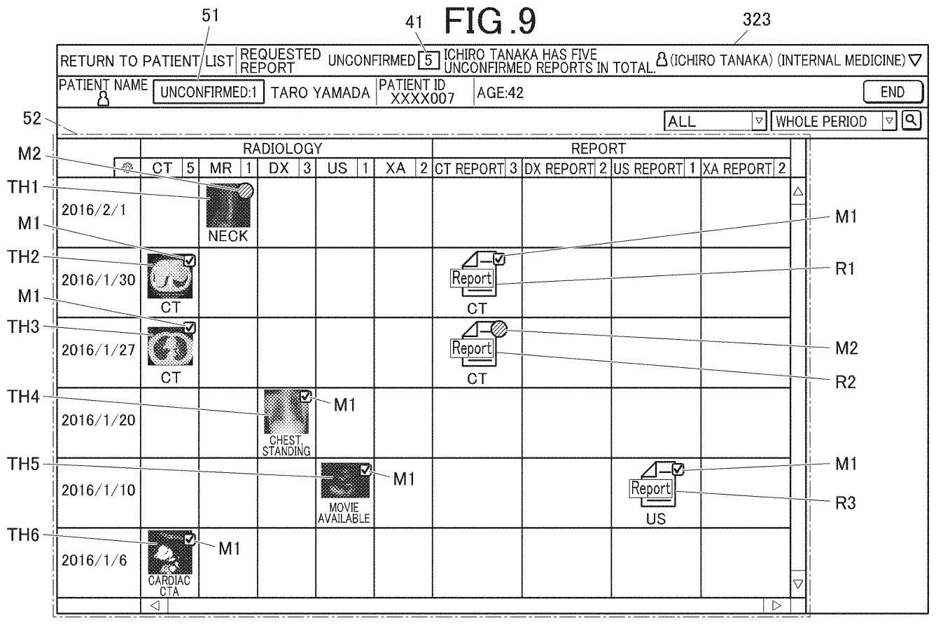

FIG. 9 is an example of an examination-and-report list window 323 displayed on the display 32 of the client terminal 30.

The examination-and-report list window 323 includes a selected-patient-unconfirmed-report-number icon 51, a matrix region 52, and a user-unconfirmed-report-number icon 41.

The selected-patient-unconfirmed-report-number icon 51 indicates the number of unconfirmed reports of the selected patient for the log-in user. When the selected-patient-unconfirmed-report-number icon 51 is pressed, the display 32 displays the list of unconfirmed reports of the selected patient for the log-in user.

More specifically, the controller 21 extracts, from the report information table T6 in the storage 24, a record(s) in which (i) the field of “patient ID” is the patient ID of the selected patient, (ii) the field of “user ID of requesting doctor” is the user ID of the log-in user, and (iii) “report confirmation status flag” is “0: unconfirmed”. The medical reports that correspond to the extracted records is the unconfirmed reports of the selected patient for the log-in user. The number of extracted records is the number of unconfirmed reports of the selected patient for the log-in user.

When the log-in user is the department head of a department, the display 32 of the client terminal 30 displays the number of unconfirmed reports requested by the department for the patient. More specifically, the controller 21 refers to the user attribution table T4 in the storage 24. When the user ID of the log-in user is associated with the authority type of “3: department head”, the controller 21 obtains the department code associated with the user ID of the log-in user. The controller 21 then extracts, from the report information table T6 in the storage 24, a record(s) in which (i) the field of “patient ID” is the patient ID of the selected patient, (ii) the field of “requesting department” is the department to which the log-in user belongs (department that corresponds to the department code obtained from the user attribution table T4), and (iii) the report confirmation status flag is “0: unconfirmed”.

The matrix region 52 displays medical reports and medical images of the selected patient in a matrix. The vertical axis of the matrix region 52 indicates the date, and the horizontal axis thereof indicates the modality type and report type. The matrix region 52 shows marks and/or images indicating medical reports and images of the selected patient in their corresponding sections.

The controller 21 extracts, from the report information table T6 in the storage 24, a record(s) in which the field of “patient ID” is the patient ID of the selected patient. The controller 21 then obtains the creation date and time, modality, and the report confirmation status flag in the extracted record. The controller 21 arranges report marks R1 to R3 in the sections corresponding to the obtained creation date and time and modality in the matrix region 52. The controller 21 also adds a confirmed mark M1 or an unconfirmed mark M2 to each of the report marks R1 to R3, according to the obtained report confirmation status flag.

In the matrix region 52, medical reports requested by the log-in user as the requesting doctor and medical reports requested by a doctor who is not the log-in user may have different unconfirmed marks M2. For example, the colors or the shapes of the unconfirmed marks M2 may be different.

The controller 21 extracts, from the medical image management table T9 stored in the storage 24, a record(s) in which the field of “patient ID” is the patient ID of the selected patient. The controller 21 then obtains the examination date and time, modality, and image check status flag in the extracted record. The controller 21 arranges thumbnail images of the medical images TH1 to TH6 in the sections corresponding to the obtained examination date and time and modality in the matrix region 52. The controller 21 also adds the confirmed mark M1 or the unconfirmed mark M2 to the thumbnail images TH1 to TH6, according to the obtained image check status flag.

The user-unconfirmed-report-number icon 41 in the examination/report list window 323 in FIG. 9 indicates the number of unconfirmed reports of the log-in user, as with the user-unconfirmed-report-number icon 41 in FIG. 7. When the user presses the user-unconfirmed-report-number icon 41, a list of unconfirmed reports of the log-in user is displayed.

After Step S8, the controller 21 of the medical information management apparatus 20 determines whether or not the number of unconfirmed reports of the selected patient for the log-in user (selected-patient-unconfirmed-report-number icon 51) displayed on the display 32 has been clicked with the operation receiver 33 of the client terminal 30 (Step S9).

When determining that the number of unconfirmed reports of the selected patient for the log-in user has been clicked (Step S9: YES), the controller 21 of the medical information management apparatus 20 causes the display 32 of the client terminal 30 to display the list of unconfirmed reports of the selected patient for the log-in user (Step S10).

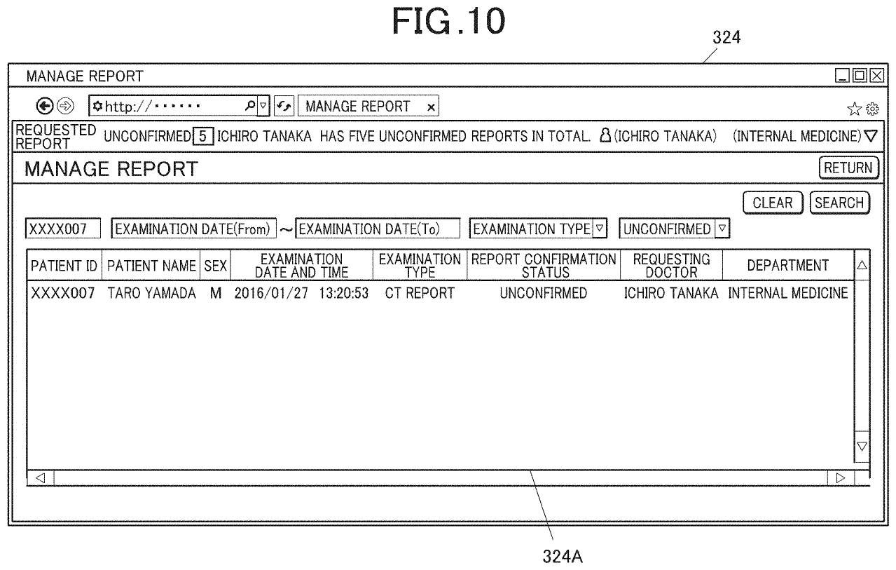

FIG. 10 is an example of an unconfirmed report list window 324 of the selected patient for the log-in user, which is displayed on the display 32 of the client terminal 30. The unconfirmed report list window 324 of the selected patient for the log-in user shows the unconfirmed report list 324A. The unconfirmed report list 324A is a list of unconfirmed reports that have been requested by the log-in user.

When the operator at the client terminal 30 selects a report in the list of unconfirmed reports of the selected patient for the log-in user with the operation receiver 33 (Step S11), the controller 21 of the medical information management apparatus 20 obtains the report ID of the selected report via the communication unit 22.

When determining that the number of unconfirmed reports of the selected patient for the log-in user has not been clicked (Step S9: NO), the controller 21 of the medical information management apparatus 20 determines whether or not a report in the list of examinations and reports of the selected patient has been selected with the operation receiver 33 of the client terminal 30 (Step S12).

When determining that a report in the list of reports and examinations of the selected patient has been selected with the operation receiver 33 of the client terminal 30 (Step S12: YES), the controller 21 of the medical information management apparatus 20 obtains the report ID of the selected report via the communication unit 22.

When determining in Step S7 that a patient has not been selected (Step S7: NO), the controller 21 of the medical information management apparatus 20 determines whether or not report search has been ordered with the operation receiver 33 of the client terminal 30 (Step S13). For example, the operator orders report search in the search window 321 shown in FIG. 7 by specifying search conditions in the search-condition specifying region 42 and pressing the search button 43.

When determining that report search has been ordered (Step S13: YES), the controller 21 of the medical information management apparatus 20 causes the display 32 of the client terminal 30 to display a list of reports that meet the search conditions (Step S14).

When the operator selects a report in the list of reports by operating the operation receiver 33 of the client terminal 30 (Step S15), the controller 21 of the medical information management apparatus 20 obtains the report ID of the selected report via the communication unit 22.

When determining in Step S12 that a report has not been selected from the list of reports and examinations of the selected patient (Step S12: NO) or determining in Step S13 that report search has not been ordered (Step S13: NO), the controller 21 returns to Step S4 and repeats the process of Step S4 and thereafter.

After Step S6, Step S11, or Step S15, or when determining in Step S12 that a report has been selected from the list of reports and examinations of the selected patient (Step S12: YES), the controller 21 performs a report window displaying process for the selected report (Step S16). The report confirmation status managing process then ends.

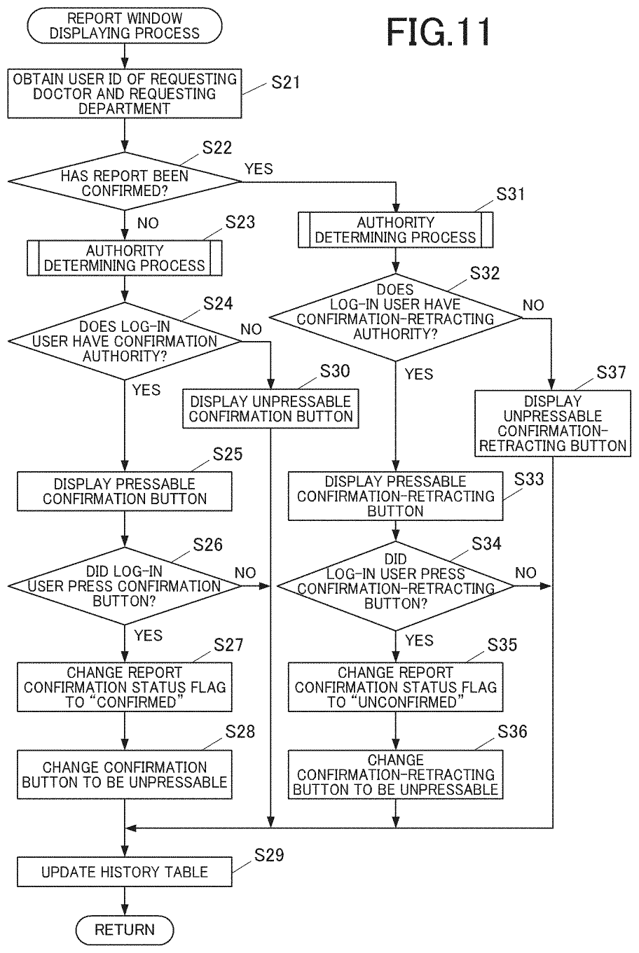

Next, the report window displaying process (Step S16) is described with reference to FIG. 11.

The controller 21 of the medical information management apparatus 20 identifies, in the report information table T6 in the storage 24, a record that corresponds to the report ID of the selected report, and obtains the user ID of requesting user and requesting department of the identified record (Step S21).

The controller 21 determines whether or not the report confirmation status flag associated with the selected record is “1: confirmed” in the report information table T6 (Step S22).

When determining that the report confirmation status flag is not “1: confirmed” (Step S22: NO), namely determining that the report confirmation status flag is “0: unconfirmed” or “2: being confirmed”, the controller 21 performs an authority determining process (Step S23). In the authority determining process in Step S23, the controller 21 determines whether or not the log-in user has confirmation authority.

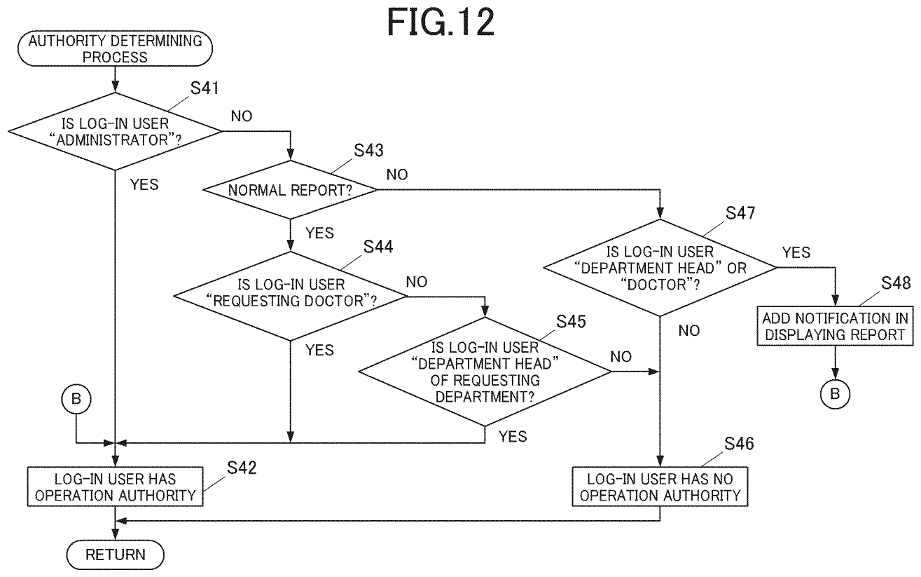

The authority determining process is described with reference to FIG. 12.

The controller 21 of the medical information management apparatus 20 determines whether or not the log-in user is the administrator (Step S41). More specifically, the controller 21 obtains the authority type associated with the user ID of the log-in user from the user attribution table T4 in the storage 24. The controller 21 then determines whether or not the obtained authority type is “4: administrator”.

When determining that the log-in user is the administrator (Step S41: YES), the controller 21 determines that the log-in user has operation authority (Step S42). When the authority determining process is for determining confirmation authority, the operation authority is confirmation authority. When the authority determining process is for determining confirmation-retracting authority, the operation authority is confirmation-retracting authority.

When determining that the log-in user is not the administrator (Step S41: NO), the controller 21 determines whether or not the selected report is a normal report (Step S43). More specifically, when the requesting department of the selected report (requesting report obtained in Step S21) is the emergency department, the controller 21 determines that the selected report is an emergency report and is not a normal report. When determining that the “requesting department” of the selected report is not the emergency department, the controller 21 determines that the selected report is a normal report. The emergency department is determined beforehand and may be changed as desired. Further, multiple departments may be designated as the emergency departments. In this embodiment, an emergency treatment department is designated as the emergency department.

When determining that the selected report is a normal report (Step S43: YES), the controller 21 determines whether or not the log-in user is the requesting doctor (Step S44). More specifically, when the user ID of requesting doctor associated with the report ID of the selected report is the user ID of the log-in user in the report information table T6, the controller 21 determines that the log-in user is the requesting doctor.

When determining that the log-in user is the requesting doctor (Step S44: YES), the controller 21 determines that the log-in user has operation authority (Step S42).

When determining that the log-in user is not the requesting doctor (Step S44: NO), the controller 21 determines whether or not the log-in user is the department head of the requesting department (Step S45). More specifically, the controller 21 refers to the user attribution table T4 in the storage 24. When finding a report in which (i) the field of “user ID” is the user ID of the log-in user, (ii) the field of “department code” is the department code of the requesting department of the selected report (department code obtained in Step S21), and (iii) the field of “authority type” is “3: department head”, the controller 21 determines that the log-in user is the department head of the requesting department.

When determining that the log-in user is the department head of the requesting department (Step S45: YES), the controller 21 determines that the log-in user has operation authority (Step S42).

When determining that the log-in user is not the department head of the requesting department (Step S45: NO), the controller 21 determines that the log-in user does not have operation authority (Step S46).

When determining in Step S43 that the selected report is not a normal report (Step S43: NO), namely determining that the selected report is an emergency report, the controller 21 determines whether or not the log-in user is a department head or a doctor (Step S47). More specifically, the controller 21 obtains the authority type associated with the user ID of the log-in user from the user attribution table T4. The controller 21 then determines whether or not the obtained authority type is “3: department head” or “2: doctor”.

When determining that the log-in user is a department head or a doctor (Step S47: YES), the controller 21 determines to add a warning in displaying the report (Step S48), and determines that the log-in user has operation authority (Step S42).

When determining in Step S47 that the log-in user is neither a department head nor a doctor (Step S47: NO), the controller 21 determines that the log-in user does not have operation authority (Step S46).

After Step S42 or Step S46, the authority determining process ends.

Referring back to FIG. 11, when determining after Step S23 that the log-in user has confirmation authority (Step S24: YES), the controller 21 of the medical information management apparatus 20 causes the display 32 of the client terminal 30 to display the report window of the selected report and the pressable confirmation button (Step S25). Herein, “pressable” means that the button can be pressed and changed into the pressed state with the operation receiver 33.

When the log-in user is given confirmation authority because the report to be displayed is an emergency report (i.e., the controller 21 has determined in Step 48 to add a warning in displaying the report), the controller 21 causes the display 32 of the client terminal 30 to display a warning message. For example, the display 32 displays “You have confirmation authority because this report is handled emergently.” or “All the doctors except resident doctors have confirmation authority.” as a warning.

Herein, the confirmation-retracting button may not be displayed or may be displayed to be unpressable. “Unpressable” means that the button cannot be pressed into the pressed state with the operation receiver 33. For example, the confirmation-retracting button may be grayed out as not operable.

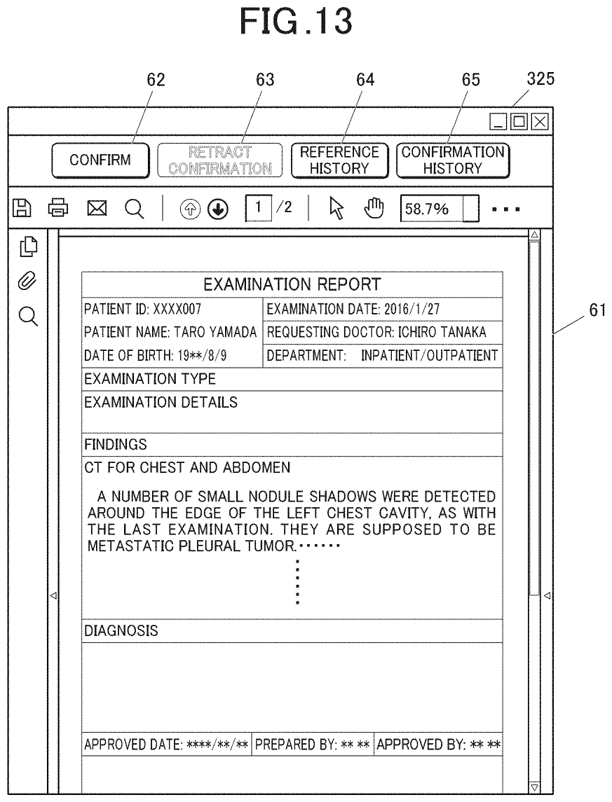

FIG. 13 is an example of the report window 325 displayed on the display 32 of the client terminal 30. The report window 325 includes a report display region 61, a confirmation button 62, a confirmation-retracting button 63, a reference-history button 64, and a confirmation-history button 65.

In the report display region 61, the selected report is displayed.

The confirmation button 62 is displayed so as to be pressable when the confirmation status of the medical report displayed in the report display region 61 is “unconfirmed” and the operator has confirmation authority. The confirmation button 62 is pressed (clicked) to determine the confirmation status of the medical report as having been confirmed.

The confirmation-retracting button 63 is displayed so as to be pressable when the confirmation status of the medical report displayed in the report display region 61 is “confirmed” and the operator has confirmation-retracting authority. The confirmation-retracting button 63 is pressed (clicked) to retract the confirmation of the medical report, (to change the confirmation status back to “unconfirmed”).

In FIG. 13, the confirmation button 62 is displayed so as to be pressable, and the confirmation-retracting button 63 is displayed so as to be unpressable.

When the reference-history button 64 is pressed, the display 32 displays a list of users who have referred to (who have displayed) the medical report displayed in the report display region 61. More specifically, the controller 21 extracts, from the report reference history table T8, a record in which (i) the field of “target report ID” is the report ID of the displayed medical report, (ii) the field of “reference/reference retraction flag” is “1: referred”. The controller 21 then displays the “referring user ID” and “report reference date and time” included in the extracted record.

When the confirmation-history button 65 is pressed, the display 32 displays a list of users who performed confirmation or retraction of confirmation on of the medical report displayed in the report display region 61. More specifically, the controller 21 extracts, from the report reference history table T8 in the storage 24, a record in which (i) the field of “target report ID” is the report ID of the displayed medical report and (ii) “confirmation status change flag” is “1: changed to “confirmed”” or “−1: confirmation retracted”. For the extracted record in which the confirmation status change flag is “1: changed to “confirmed””, the controller 21 displays the referring user ID and the report confirmation date and time in the record as the user who confirmed the medical report and the date and time at which the user confirmed the medical report. For the extracted record in which the confirmation status change flag is “−1: confirmation retracted”, the controller 21 displays the referring user ID and the report reference date and time in the record as the user who retracted the confirmation of the medical report and the date and time at which the user retracted the confirmation of the medical report.

When the operator presses the confirmation button 62 by operating the operation receiver 33 of the client terminal 30 (Step S26: YES), the controller 21 of the medical information management apparatus 20 changes the report confirmation status flag associated with the report ID of the selected report into “1:confirmed” in the report information table T6 (Step S27).

The controller 21 changes the confirmation button 62 on the display 32 of the client terminal 30 into the unpressable state, thereby prohibiting the operator from pressing the confirmation button 62 again (Step S28).

The controller 21 updates the report confirmation history table T7 and the report reference history table T8 in the storage 24 (Step S29).

More specifically, the controller 21 associates the target report ID with the confirming user ID, report confirmation date and time, and confirmation authority type, and stores the associated information in the report confirmation history table T7. The controller 21 stores (i) the report ID of the selected report in the field of “target report ID”, (ii) the user ID of the log-in user in the field of “confirming user ID”, (iii) the current date and time obtained from the timer 23 in the field of “report confirmation date and time”, and (iii) the authority type associated with the user ID of the log-in user in the user attribution table T4 in the field of “confirmation authority type”.

Further, the controller 21 adds a new record in the report reference history table T8. The controller 21 stores (i) the report ID of the selected report in the field of “target report ID”, (ii) the user ID of the log-in user in the field of “referring user ID”, (iii) the current date and time obtained from the timer 23 in the field of “report reference date and time”, and (iv) the authority type associated with the user ID of the log-in user in the user attribution table T4 in the field of “reference authority type”. For the new record, the controller 21 sets “reference/reference retraction flag” to be “1: referred” and “confirmation status change flag” as “1: changed to “confirmed””.

When determining in Step S24 that the log-in user does not have confirmation authority (Step S24: NO), the controller 21 of the medical information management apparatus 20 causes the display 32 of the client terminal 30 to display the report window of the selected report and the confirmation button 62 that cannot be pressed (Step S30).

Herein, the confirmation-retracting button 63 may not be displayed or may be displayed so as to be unpressable.

After Step S30 or when determining in Step S26 that the confirmation button 62 has not been pressed (Step S26: NO), the controller 21 updates the report reference history table T8 in the storage 24 (Step S29).

More specifically, the controller 21 adds a new record in the report reference history table T8. For the new record, the controller 21 stores (i) the report ID of the selected report in the field of “target report ID”, (ii) the user ID of the log-in user in the field of “referring user ID”, (iii) the current date and time obtained from the timer 23 in the field of “report reference date and time”, and (iv) the authority type associated with the user ID of the log-in user in the user attribution table T4 in the field of “reference authority type”. For the new record, the controller 21 also sets the reference/reference retraction flag to “1: referred” and the confirmation status change flag to “0: unchanged”.

When determining in Step S22 that the report confirmation status flag of the selected report is “1: confirmed” (Step S22: YES), the controller 21 performs the authority determining process shown in FIG. 12 (Step S31). The authority determining process in Step S31 is the same as the authority determining process in Step S23 except that the operation authority in Step S31 is the confirmation-retracting authority. That is, when a doctor is given confirmation authority of medical information stored in the storage 24, the controller 21 permits the doctor to retract the confirmation of the medical report.

After Step S31, when determining that the log-in user has confirmation-retracting authority (Step S32: YES), the controller 21 of the medical information management apparatus 20 causes the display 32 of the client terminal 30 to display the report window 325 of the selected report and the confirmation-retracting button 63 that can be pressed (Step S33).

When the log-in user is given confirmation-retracting authority because the report to be displayed is an emergency report (i.e., the controller 21 has determined in Step S48 to add a warning in displaying the report), the controller 21 causes the display 32 of the client terminal 30 to display a warning message. For example, the display 33 displays “You have confirmation-retracting authority because this report is handled emergently.” or “All the doctors except resident doctors have confirmation-retracting authority.” as a warning.

The confirmation button 62 may not be displayed or may be displayed so as to be unpressable.

When the operator presses the confirmation-retracting button 63 by operating the operation receiver 33 of the client terminal 30 (Step S34: YES), the controller 21 of the medical information management apparatus 20 changes, in the report information table T6 in the storage 24, the report confirmation status flag associated with the report ID of the selected report into “0: unconfirmed” (Step S35).

The controller 21 also changes, in the report window 325 in the display 32 of the client terminal 30, the confirmation-retracting button 63 to be unpressable, thereby prohibiting the operator from pressing the confirmation-retracting button 63 (Step S36).

The controller 21 updates the report reference history table T8 in the storage 24 (Step S29).

More specifically, the controller 21 adds a new record in the report reference history table T8. For the new record, the controller 21 stores (i) the report ID of the selected report in the field of “target report ID”, (ii) the user ID of the log-in user in the field of “referring user ID”, (iii) the current date and time obtained from the timer 23 in the field of “report reference date and time”, and (iv) the authority type associated with the user ID of the log-in user in the user attribution table T4 in the field of “reference authority type”. For the new record, the controller 21 also sets the reference/reference retraction flag to “1: referred” and the confirmation status change flag to “−1: confirmation retracted”.

When determining in Step S32 that the log-in user does not have confirmation-retracting authority (Step S32: NO), the controller 21 of the medical information management apparatus 20 causes the display 32 of the client terminal 30 to display the report window 325 of the selected report and the confirmation-retracting button 63 that cannot be pressed (Step S37).

The confirmation button 62 may not be displayed or may be displayed so as to be unpressable.

After Step S37 or when determining in Step S34 that the confirmation-retracting button 63 has not been pressed (Step S34: NO), the controller 21 updates the report reference history table T8 in the storage 24 (Step S29).

More specifically, the controller 21 adds a new record in the report reference history table T8. For the new record, the controller 21 stores (i) the report ID of the selected report in the field of “target report ID”, (ii) the user ID of the log-in user in the field of “referring user ID”, (iii) the current date and time obtained from the timer 23 in the field of “report reference date and time”, and (iv) the authority type associated with the user ID of the log-in user in the user attribution table T4 in the field of “reference authority type”. For the new record, the controller 21 also sets the reference/reference retraction flag to “1: referred” and the confirmation status change flag to “0: unchanged”.

After Step S29, the controller 21 ends the report window displaying process.

According to the embodiment described above, when target medical information is associated with identification information of a doctor operating the client terminal 30 in the report information table T6 in the storage 24, the doctor is given confirmation authority to determine the confirmation status of the target medical information as having been confirmed. Thus, a doctor who is not supposed to confirm the medical information is prevented from determining the confirmation status of the medical information as having been confirmed. Medical information can therefore be managed securely.

For example, the display 32 of the client terminal 30 displays a confirmation button for determining the confirmation status of the target medical information as having been confirmed such that the confirmation button is able to be pressed by operating the operation receiver 33. This allows the operator of the client terminal 30 to recognize that the operator has the confirmation authority to determine the confirmation status of the target medical information as having been confirmed.

Further, when the confirmation button displayed on the display 32 of the client terminal 30 is pressed by operating the operation receiver 33, the controller 21 changes the confirmation status of the target medical information in the report information table T6 in the storage 24 as having been confirmed, and prohibits the confirmation button from being pressed again. The medical information is thus shown as having been confirmed.

Further, when the target medical information is not associated with the doctor identification information of the doctor operating the client terminal 30 in the report information table T6 in the storage 24, the controller 21 causes the display 32 of the client terminal 30 to display the confirmation button such that the confirmation button cannot be pressed by operating the operation receiver 33. The controller 21 thus easily shows that the operator does not have confirmation authority.

Further, when the target medical information is emergency medical information that is handled emergently and is not associated with the doctor identification information of the doctor operating the client terminal 30 in the report information table T6 of the storage 24, the controller 21 gives the confirmation authority of the target medical information to the doctor. For example, an emergency department may not determine a doctor who confirms the report when ordering a medical report. By giving confirmation authority to all the doctors, the medical report can be smoothly handled in the subsequent process.

Further, in giving the confirmation authority of the target medical information to the doctor due to the target medical information being the emergency medical information, the controller 21 causes the display 32 of the client terminal 30 to display a warning. The controller 21 thus can call attention of the operator. More specifically, the operator can be aware that the confirmation authority of the target medical information is given to all the doctors and can distinguish medical information the confirmation authority of which is given only to the operator from the emergency medical information.

Further, as shown in the unconfirmed report list window 322 for the log-in user in FIG. 8, when the controller 21 causes the display 32 of the client terminal 30 to display a list of unconfirmed medical information for which confirmation authority is given to the doctor operating the client terminal 30 and the confirmation status is a status of not having been confirmed, the controller 21 excludes, from the list, the medical information the confirmation authority of which is given to the doctor due to the medical information being the emergency medical information. This allows the operator to easily recognize medical information the confirmation authority of which is given only to the operator (medical information requested by the log-in user as the requesting doctor).

Further, when the doctor operating the client terminal 30 is in charge of a department to which another doctor belongs, the another doctor being indicated by doctor identification information associated with the target medical information, the controller 21 gives the confirmation authority to the doctor operating the client terminal 30. Accordingly, the medical information can be confirmed even when a doctor who is supposed to confirm the medical information is absent due to retirement or temporary leave.

Further, the controller 21 records a history of determining the confirmation status of the target medical information as having been confirmed, so that the confirmation status of medical information can be managed.

Further, the controller 21 permits a doctor that is given confirmation authority of the medical information to retract confirmation of the medical information. This can prevent a person who does not have confirmation authority from retracting the confirmation.

The embodiment described above is one of preferred examples of the medical information management system according to the present invention, and is not intended to limit the present invention. The detailed configuration and detailed operation of the components constituting the medical information management system can be appropriately modified without departing from the scope of the present invention.

For example, although medical reports are mainly confirmed in the above embodiment, other types of medical information may be confirmed.

Further, in the above embodiment, although the control program of the present invention is performed by the medical information management apparatus 20 in the medical information management system 100, the control program may be performed by other apparatuses.

Further, in the above embodiment, confirmation authority and confirmation-retracting authority are determined through the same authority determining process (FIG. 12). However, the confirmation authority and confirmation-retracting authority, which are determined based on the authority type of the user, may be determined differently.

Further, in the above embodiment, the user consciously determines that medical information (medical report) is confirmed. Alternatively, the medical information may be automatically set into the confirmed status. For example, the medical report may be automatically set into the confirmed status when the report is detected as having been displayed and viewed (referred). The medical report may also be automatically set in the confirmed status after the medical report is displayed and viewed for a certain period of time.

In the above description, the ROM is used as a computer readable medium that stores the programs for performing the processes. However, the computer readable medium is not limited thereto. As the other computer-readable storage medium, a nonvolatile memory, such as a flash memory, and a portable storage medium, such as a CD-ROM, may also be used. Further, as a medium to provide data of the programs via a communication line, a carrier wave can be used.

Although embodiments of the present invention have been described and illustrated in detail, the disclosed embodiments are made for purposes of illustration and example only and not limitation. The scope of the present invention should be interpreted by terms of the appended claims.

What is claimed is:

1. A medical information management apparatus, comprising:
a storage that stores medical information, doctor identification information, and a confirmation status of the medical information that are associated with each other; and
a hardware processor that:
obtains doctor identification information of a doctor that operates a display;
in displaying target medical information on the display, in response to the target medical information being associated with the obtained doctor identification information in the storage, gives confirmation authority to the doctor, the confirmation authority being authority to determine a confirmation status of the target medical information in the storage as having been confirmed; and
provides the display with a confirmation instruction receiver for determining the confirmation status of the target medical information as having been confirmed.

2. The medical information management apparatus according to claim 1, wherein
the hardware processor causes the display to display, as the confirmation instruction receiver, a confirmation button for determining the confirmation status of the target medical information as having been confirmed such that the confirmation button is able to be pressed by operating an operation receiver.

3. The medical information management apparatus according to claim 2, wherein
in response to the confirmation button displayed on the display being pressed by operating the operation receiver, the hardware processor
changes the confirmation status of the target medical information in the storage as having been confirmed and
prohibits the confirmation button from being pressed again.

4. The medical information management apparatus according to claim 2, wherein
in response to the target medical information not being associated with the obtained doctor identification information in the storage, the hardware processor causes the display to display the confirmation button such that the confirmation button is unable to be pressed by operating the operation receiver.

5. The medical information management apparatus according to claim 1, wherein
in response to the target medical information being emergency medical information that is handled emergently and not being associated with the obtained doctor identification information in the storage, the hardware processor gives the confirmation authority to the doctor.

6. The medical information management apparatus according to claim 5, wherein
in giving the confirmation authority of the target medical information to the doctor due to the target medical information being the emergency medical information, the hardware processor causes the display to display a warning.

7. The medical information management apparatus according to claim 5, wherein
the hardware processor causes the display to display a list of unconfirmed medical information for which confirmation authority is given to the doctor indicated by the obtained doctor identification information and a confirmation status is a status of not having been confirmed, and
the hardware processor excludes, from the list, the medical information the confirmation authority of which is given to the doctor due to the medical information being the emergency medical information.

8. The medical information management apparatus according to claim 1, wherein
the doctor indicated by the obtained doctor identification information is in charge of a department to which another doctor belongs, the another doctor being indicated by doctor identification information associated with the target medical information in the storage, and
the hardware processor gives the confirmation authority to the doctor indicated by the obtained doctor identification information.

9. The medical information management apparatus according to claim 1, wherein
the hardware processor records a history of determining the confirmation status of the target medical information as having been confirmed with the confirmation instruction receiver.

10. The medical information management apparatus according to claim 1, wherein
the hardware processor permits a doctor that is given confirmation authority of the medical information stored in the storage to retract confirmation of the medical information.

11. The medical information management apparatus according to claim 1, wherein
the medical information is a medical report, and
in the storage, the medical report is associated with the doctor identification information of a doctor that has requested creation of the medical report.

12. A medical information management system, comprising:
a storage that stores medical information, doctor identification information, and a confirmation status of the medical information that are associated with each other;
a display; and
a hardware processor that:
obtains doctor identification information of a doctor that operates the display;
in displaying target medical information on the display, in response to the target medical information being associated with the obtained doctor identification information in the storage, gives confirmation authority to the doctor, the confirmation authority being authority to determine a confirmation status of the target medical information in the storage as having been confirmed; and
provides the display with a confirmation instruction receiver for determining the confirmation status of the target medical information as having been confirmed.

13. A non-transitory computer readable storage medium storing a program to cause a computer of a medical information management apparatus that includes a storage storing medical information, doctor identification information, and a confirmation status of the medical information that are associated with each other to function as a hardware processor that:
obtains doctor identification information of a doctor that operates the display;
in displaying target medical information on the display, in response to the target medical information being associated with the obtained doctor identification information in the storage, gives confirmation authority to the doctor, the confirmation authority being authority to determine a confirmation status of the target medical information in the storage as having been confirmed; and
provides the display with a confirmation instruction receiver for determining the confirmation status of the target medical information as having been confirmed.

14. A medical information management method for a medical information management apparatus that includes a storage storing medical information, doctor identification information, and a confirmation status of the medical information that are associated with each other, the method comprising:
obtaining doctor identification information of a doctor that operates a display;
in displaying target medical information on the display, in response to the target medical information being associated with the obtained doctor identification information in the storage, giving confirmation authority to the doctor, the confirmation authority being authority to determine a confirmation status of the target medical information in the storage as having been confirmed; and
providing the display with a confirmation instruction receiver for determining the confirmation status of the target medical information as having been confirmed.
